(19) United States
(12) Patent Application Publication
(10) Pub. No.: US 2008/0280778 A1
(43) Pub. Date: Nov. 13, 2008

(54) Binding reagents that contain small epitope binding molecules

(76) Inventor: Michael S. Urdea, Alamo, CA (US)

Correspondence Address: Tethys Bioscience;c/o Morrison & Foerster LLP, 755 Page Mill Road, Palo Alto, CA, 94304-1018, US

(21) Appl. No.: 12/151,220

(22) Filed: May 2, 2008

Related U.S. Application Data
(60) Provisional application No. 60/927,656, filed on May 3, 2007.

(51) Int. Cl. C40B 30/04; G01N 33/543; C07K 1/00; C07H 1/00; C40B 40/00

(52) U.S. Cl. 506/9; 436/518; 506/13; 530/413; 536/25.4

(57) ABSTRACT

The invention provides binding reagents that contain a plurality of linked small epitope binding molecules that each recognizes a small epitope, such as small epitope antibodies. The combination of small epitope binding molecules in a binding reagent specifically recognizes and binds to a molecule of interest. The binding reagents may be used for such purposes as detection, quantification, identification, and purification of molecules of interest.

### CROSS-REFERENCE TO RELATED APPLICATIONS

This application claims the priority benefit of provisional application U.S. Ser. No. 60/927,656, filed May 3, 2007, the contents of which are incorporated by reference in the entirety.

### FIELD OF THE INVENTION

The present invention relates generally to binding reagents that contain a plurality of linked small epitope binding molecules, and use thereof in methods for analysis of molecules containing small epitopes recognized by the small epitope binding molecules.

### BACKGROUND OF THE INVENTION

There is a great need to produce reagents for the specific binding to molecules of interest. Analysis of one or more molecules in a mixture, including determination of presence, absence, or amount of such molecules, is often performed using an immunoassay, such as an enzyme-linked immunosorbent assay (ELISA). ELISA techniques are widely used in diagnostic assessments, such as analysis of one or more proteins or metabolites in serum or plasma.

Immunoassays require development of antibodies that bind specifically to each molecule of interest to be assayed. However, production of polyclonal or monoclonal antibodies is time consuming and often requires multiple immunizations of an animal and an amount of purified immunogen that may be difficult to obtain. Further, the binding quality of the antibody reagents obtained depends on the antibody repertoire of the animal being immunized. It would be desirable to be able to produce a binding reagent that could be assembled based on knowledge of the structure of the molecule to be assayed and without the need for animal immunization, which often results in reagents of varying specificity. Attempts have been made to produce other types of molecules capable of binding proteins (e.g., molecular imprints and aptamers) in order to hasten or circumvent antibody production. These molecules have been shown to possess limited utility. There is a need for improved methods for producing binding reagents that can specifically bind to molecules of interest, for example, for use in immunoassays, immunodiagnostics, in vivo imaging, or immunodetection, or for use as immunotherapeutic agents, therapeutic targeting agents, affinity capture reagents, or affinity agents in affinity chromatography.

### BRIEF SUMMARY OF THE INVENTION

In one aspect, the invention provides a binding reagent comprising a plurality of small epitope binding molecules, wherein said small epitope binding molecules are linked, and wherein each of said small epitope binding molecules recognizes a different small epitope. In one embodiment, the plurality of small epitope binding molecules consists of 2, 3, or 4 small epitope binding molecules. In one embodiment, the binding reagent binds specifically to a polypeptide. In other embodiments, the binding reagent binds specifically to a polynucleotide, a polysaccharide, a glycoprotein, a lipid, a lipoprotein, a post-translationally modified protein, or a polynucleotide comprising one or more modified nucleotide bases. In some embodiments, the binding reagent comprises a detectable label.

In some embodiments, the small epitope binding molecules are linked covalently. In one embodiment, the covalent linkage comprises a chemical cross-linking reagent. In some embodiments, the small epitope binding molecules are linked by interaction between members of a binding pair.

In some embodiments, the binding reagent is recombinantly expressed as a polypeptide with a peptide linker between the antibodies or epitope binding fragments. In some embodiments, the small epitope binding molecules of the binding reagent are small epitope antibodies or epitope binding fragments thereof. In one embodiment, each small epitope antibody or epitope binding fragment thereof recognizes an epitope consisting of 3, 4, or 5 contiguous amino acids. In one embodiment, the plurality of small epitope binding molecules consists of 2, 3, or 4 small epitope antibodies or epitope binding fragments thereof.

In some embodiments, the small epitope binding molecules are selected from the group consisting of aptamers, molecular imprints, lectins, and capture compounds.

In one embodiment, the invention provides a library of binding reagents, comprising a plurality of binding reagents as described above, wherein each of said binding reagents recognizes a different combination of small epitopes.

In one embodiment, the invention provides an array comprising a plurality of binding reagents as described above immobilized on a support, wherein each of said binding reagents recognizes a different combination of small epitopes.

In another aspect, the invention provides a method of characterizing a molecule of interest, said method comprising contacting a sample suspected of comprising the molecule of interest with a binding reagent as described above, wherein said binding reagent is capable of specifically binding to the molecule of interest, and detecting binding of the molecule of interest to the binding reagent. In one embodiment, characterizing comprises determining the amount of the molecule of interest in the sample. In on embodiment, characterizing comprises determining presence or absence of the molecule of interest in the sample. In one embodiment, the molecule of interest is a biomarker.

In another aspect, the invention provides a method of making a binding reagent as described above. In one embodiment, the method comprises linking said small epitope binding molecules with a chemical cross-linking reagent. In another embodiment, the method comprises linking said small epitope binding molecules via interaction between members of a binding pair. In another embodiment, the method comprises recombinant expression of a polypeptide comprising said small epitope antibodies or epitope binding fragments thereof.

In another aspect, the invention provides a kit comprises a binding reagent as described above.

In another aspect, the invention provides a composition comprising a binding reagent as described above. In one embodiment, the composition is a pharmaceutical composition comprising a binding reagent and a pharmaceutically acceptable carrier.

In another aspect, the invention provides an affinity matrix comprising a binding reagent as described above attached to a substrate. In one embodiment, the invention provides a method for purifying a molecule of interest, comprising contacting the affinity matrix with a sample comprising the molecule of interest, wherein said binding reagent binds to said molecule of interest. In some embodiments, the molecule of interest is selected from the group consisting of a polypeptide, a polynucleotide, a polysaccharide, and a glycoprotein.

### BRIEF DESCRIPTION OF THE DRAWINGS

FIG. 1 schematically depicts several methods for preparation of binding reagents as described herein. FIG. 1A depicts chemical cross-linking of small epitope antibodies (“DA1” and “DA2”). FIG. 1B depicts recombinant expression of linked small epitope antibodies. FIG. 1C depicts linkage of small epitope antibodies through interaction by members of a binding pair. FIG. 1D depicts chemical cross-linking of aptamers. FIG. 1E depicts preparation of linked aptamers via polynucleotide synthesis. FIG. 1F depicts recombinant expression of linked aptamers.

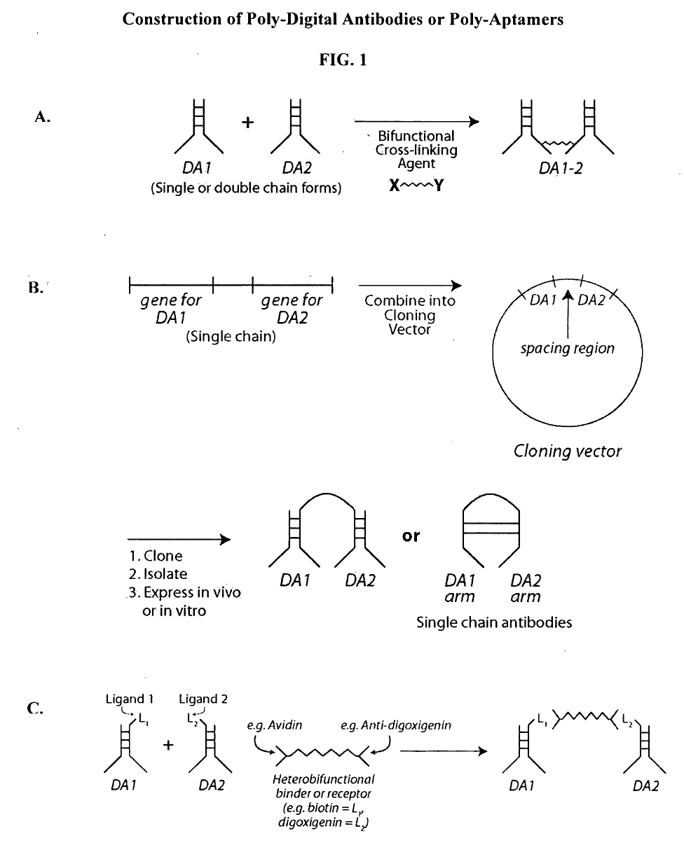

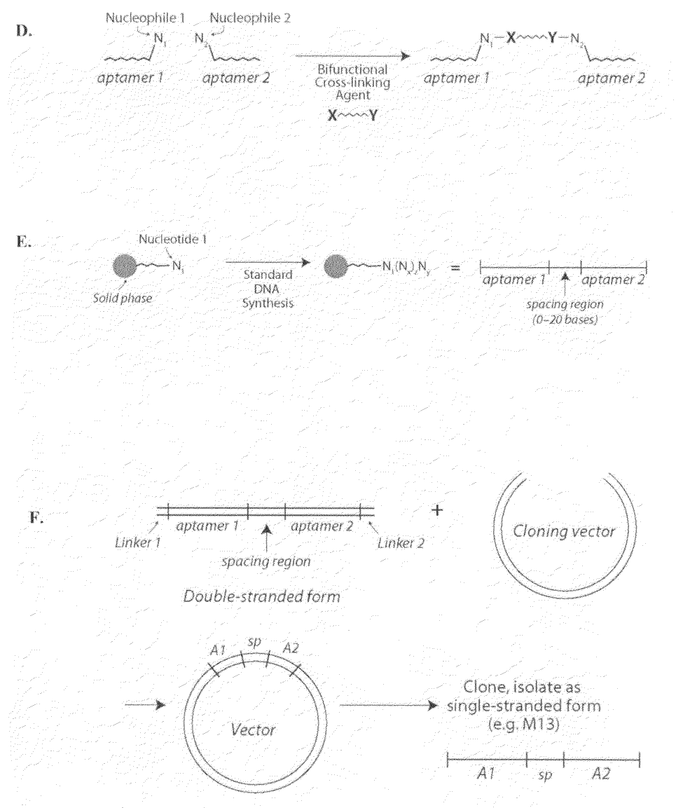

### DETAILED DESCRIPTION OF THE INVENTION

The invention provides binding reagents capable of specifically binding to molecules of interest. A “binding reagent” as described herein comprises a plurality of linked small epitope binding molecules, and a molecule recognized by a binding reagent comprises small epitopes recognized and specifically bound by the small epitope binding molecules. The binding reagents of the invention may bind to polymeric biomolecules such as polypeptides (including polypeptides comprising post-translational modifications), polysaccharides, or polynucleotides. The invention also provides compositions and kits comprising the binding reagents of invention, methods of making the binding reagents, and methods of using the binding reagents to characterize, enrich, or purify one or more molecules of interest in a sample.

A binding reagent of the invention comprises a plurality of linked small epitope binding molecules, each of which recognizes a different small epitope. The combination of small epitope binding molecules in the binding reagent collectively and specifically binds to a molecule of interest, e.g., a polypeptide, polysaccharide, or nucleic acid, or combination thereof (e.g., a glycoprotein), that contains the combination of small epitopes in an orientation that is accessible to small epitope binding sites in the binding reagent. Each small epitope binding molecule in a binding reagent of the invention is incapable of uniquely identifying the molecule of interest by virtue of binding to the small epitope recognized by the small epitope binding molecule therein, but the binding reagent is capable of unique identification of the molecule of interest by virtue of binding of the combination of small epitope binding molecules thereto.

In one embodiment, the invention provides a library of small epitope binding molecules from which may be selected a subset of small epitope binding molecules to produce a binding reagent that binds to a molecule of interest that comprises small epitopes recognized by the selected small epitope binding molecules. The small binding reagent is produced by linking the selected small epitope binding molecules together.

General Techniques

The practice of the present invention will employ, unless otherwise indicated, conventional techniques of molecular biology (including recombinant techniques), microbiology, cell biology, biochemistry, and immunology, which are within the skill of the art.

### DEFINITIONS

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion, and any other modified configuration of the immunoglobulin molecule that comprises an antigen recognition site of the required specificity. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

“Fv” is an antibody fragment that contains a complete antigen-recognition and -binding site. In a two-chain Fv species, this region consists of a dimer of one heavy and one light chain variable domain in tight, non-covalent association. In a single-chain Fv species, one heavy and one light chain variable domain can be covalently linked by a flexible polypeptide linker such that the light and heavy chains can associate in a dimeric structure analogous to that in a two-chain Fv species. It is in this configuration that the three CDRs of each variable domain interact to define an antigen-binding specificity on the surface of the VH-VL dimer. However, even a single variable domain (or half of a Fv comprising only 3 CDRs specific for an antigen) has the ability to recognize and bind antigen, although generally at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge regions.

A “monoclonal antibody” refers to a homogeneous antibody preparation wherein the monoclonal antibody is comprised of amino acids (naturally occurring and non-naturally occurring) that are involved in the selective binding of an antigen. Monoclonal antibodies (as opposed to polyclonal antibodies) are highly specific, in the sense that they are directed against a single antigenic site. The term “monoclonal antibody” encompasses not only intact monoclonal antibodies and full-length monoclonal antibodies, but also fragments thereof (such as Fab, Fab′, F(ab′)₂, Fv), single chain (ScFv), mutants thereof, fusion proteins comprising an antibody portion, and any other modified configuration of the immunoglobulin molecule that comprises an antigen recognition site of the required specificity and the ability to bind to an antigen (see definition of antibody). It is not intended to be limited as regards to the source of the antibody or the manner in which it is made (e.g., by hybridoma, phage selection, recombinant expression, transgenic animals, etc.).

The terms “polypeptide,” “oligopeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention, for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

An epitope that “specifically binds” or “preferentially binds” (used interchangeably herein) to an antibody is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an epitope is an antibody that binds this epitope with greater affinity, avidity, more readily, and/or with greater duration than it binds to other epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “sample” encompasses a variety of sample types, including those obtained from an individual. The definition encompasses blood and other liquid samples of biological origin, solid tissue samples such as a biopsy specimen or tissue cultures or cells derived therefrom, and the progeny thereof. A sample can be from a microorganism (e.g., bacteria, yeasts, viruses, viroids, molds, fungi) plant, or animal, including mammals such as humans, rodents (such as mice and rats), and monkeys (and other primates). A sample may comprise a single cell or more than a single cell. The definition also includes samples that have been manipulated in any way after their procurement, such as by treatment with reagents, solubilization, or enrichment for certain components, such as proteins or polynucleotides. The term “sample” encompasses a clinical sample, and also includes cells in culture, cell supernatants, cell lysates, serum, plasma, biological fluid, human tissue propagated in animals, and tissue samples. Examples of a sample include blood, plasma, serum, urine, stool, cerebrospinal fluid, synovial fluid, amniotic fluid, saliva, lung lavage, semen, milk, nipple aspirate, prostatic fluid, mucous, and tears.

The “complexity” of a sample means the number of different protein species, including number of different proteins as well as number of different protein variants (including splice variants, polymorphisms, and protein degradation products).

“Detect” refers to identifying (determining) the presence, absence and/or amount of the object or substance to be detected, and as described herein, detection may be qualitative and/or quantitative.

An “aptamer” refers to a nucleic acid, e.g., RNA, DNA, or modified RNA or DNA, that specifically binds to a target molecule, e.g., amino acid, carbohydrate, antibiotics, protein, by virtue of the three-dimensional structure of the nucleic acid, which provides specific contact points for interaction with the target molecule.

A small epitope binding aptamer may be isolated for a particular small epitope target using a variety of techniques. In general, aptamer discovery is based on the Systematic Evolution of Ligands by Exponential Enrichment (SELEX) technology, an in vitro selection process that isolates specific sequences (aptamers) that bind with high affinity to a given target from extremely large random oligonucleotide sequence pools (libraries) (REFERENCES 1-2). Typically this is done by introducing the library of oligonucleotides to a target molecule and then isolating those oligonucleotides that bind to the target (aptamers) from those that do not bind to the target. Typically the target is attached to a solid phase or has some other handle to facilitate partitioning the bound aptamer-target complex from the unbound oligonucleotides. The partitioning of the aptamers is followed by amplification, usually by PCR, and the process is typically repeated several times to enrich the population for the best aptamers. For small epitope aptamers the targets may consist of small epitope targets, such as peptides, 3, 4 or 5 amino acids in length, either bound to a solid surface or labeled in such a way as to facilitate aptamer-target complex partitioning. Other methods may be used to select for affinity or specificity such as standard negative selection methods where one or more rounds of selection are performed using non-small epitope containing oligomers and those aptamers that bind to these decoys are removed. Polyanionic species such as heparin or dextran sulfate may be added with or without significant dilution following initial aptamer binding to compete out low affinity interactions. Other methods could use multiple different targets that are all different in overall composition but display the same target small epitope in different sequence contexts. In an approach such as this, a different target oligomer containing the target small epitope might be used from one round of selection to the next. In another method the oligomer containing the target small epitope may be combined with one or more decoy oligomers. These decoy oligomers would not contain the target small epitope and would not be attached to a solid support or labeled to facilitate partitioning such that aptamer-decoy oligomer complexes would not be selected for. Multiple aptamers specific to different small epitopes may be selected for in the same SELEX experiment by using oligomers that contain the multiple different small epitopes in different sequence order such that the sequence context of each small epitope is different in each oligomer. One or more different oligomers could be used one at a time in successive rounds of SELEX such that the aptamers are presented with a different sequence context for the small epitopes each round. Alternatively, multiple oligomers could be used each round such that those aptamers specific for the small epitope see an effectively higher concentration of target than those aptamers that recognize undesired epitopes (discussed in Tuerk et al. (1990). Science. 249: 505-510; and Ellington et al. (1990) Nature 346: 818-822).

As used herein, the singular form “a”, “an”, and “the” includes plural references unless indicated otherwise. For example, “an” antibody includes one or more antibodies and “a protein” means one or more proteins.

“Microarray” and “array,” as used interchangeably herein, comprises a surface with an array, preferably ordered array, of putative binding sites for proteins.

Binding Reagents

The invention provides binding reagents that recognize and specifically bind to molecules of interest, such as polypeptides, polysaccharides, or polynucleotides, or a combination thereof. A binding reagent of the invention comprises a plurality of linked small epitope binding molecules each of which recognizes a different small epitope. A binding reagent of the invention specifically binds to a target molecule of interest that contains a combination of small epitopes recognized by the small epitope binding molecules in the binding reagent. In some embodiments, the target molecule recognized by a binding reagent of the invention is a polypeptide, and the small epitopes recognized by the small epitope binding molecules consist of 3, 4, or 5 amino acids within the polypeptide.

The binding reagents of the invention may be used in applications such as methods for detection and/or quantification of one or more molecules of interest in a sample, expression profiling, monitoring expression of one or more proteins over time, detection and/or quantification of biomarkers, including assessment of changes in biomarker levels over time, detection and/or quantitation and/or monitoring of expression of different isoforms or modified forms of a protein of interest, and enrichment and/or purification of a molecule of interest, e.g., affinity chromatography or other methods to separate a molecule of interest bound to a binding reagent of the invention from other molecules in a sample.

As used herein, a “small epitope binding molecule” refers to a molecule that recognizes and is capable of specifically binding to a small epitope within a target molecule of interest. A binding reagent of the invention contains small epitope binding molecules each of which is incapable of specifically identifying the target molecule by virtue of its binding alone without the associated binding of the other small epitope binding molecules in the binding reagent. The combination of small epitope binding molecules in a binding reagent imparts specificity of recognition and binding with respect to the target molecule.

Small epitope binding molecules include, but are not limited to, antibodies (i.e., small epitope antibodies), aptamers, molecular imprints (Mosbach (1994) Trends Biochem sci 19(1):9-14; Nicholls et al. (1995) Trends Biotechnol 12(2):47-51; Andersson et al. (1995) Proc Natl Acad Sci 92(11):4788-92; Mosbach (2006) Sci Am 295(4):86-91), lectins, capture compounds (e.g., described in U.S. Application No. 2004/0209255 and PCT Application Nos. WO 03/077851, WO 03/092581, and WO 04/064972), or Protein A binding domains (Nat Biolechnol (2005) 23(12):1556-61).

In some embodiments, small epitope binding molecules contain alternative (i.e., non-immunoglobulin) scaffolds. Examples of such alternative scaffolds include anticalins, derived from a lipocalin lipid binding pocket (Beste et al. (1999) Proc Natl Acad Sci 96:1898-1903); Skerra (2000) Biochimica et Biophysica Acta 1842:337-50; Weiss and Lowman (2000) Chemistry and Biology 7(8): R177-R184) and T cell receptors (Holler et al. (2000) Proc Natl Acad Sci 97: 5387-92; Kieke et al. (1999) Proc Natl Acad Sci 96:5651-56).

A small epitope recognized by a small epitope binding molecule may be a monomer (e.g., an amino acid, a nucleotide, or a carbohydrate molecule), a part of a monomer, or a short stretch of monomeric units within a biopolymer. The small epitope is specifically recognized by the small epitope binding molecule but is too small to specifically identify the biopolymer by virtue of binding of the small epitope binding molecule. For example, a small epitope binding molecule may specifically recognize a nucleotide monomer, for example, adenine, guanine, thymine, cytosine, or uracil, or short stretch of nucleotides (e.g. 2 to 5_nucleotides), a carbohydrate monomer, i.e., a monosaccharide residue, such as, for example, glucose, fructose, mannose, glucosamine, galactose, or galactosamine, a short stretch of monosaccharide molecules, (e.g., 2 to 5 carbohydrate monomers), or a particular polysaccharide branching configuration. In some embodiments, the small epitope binding molecules are “small epitope antibodies,” which specifically recognize a “small epitope” consisting of or consisting essentially of 3, 4, or 5 amino acids, within a protein. In some embodiments, a binding reagent as described herein may contain small epitope binding molecules that recognize different types of monomeric units. For example, a binding reagent may contain one or more small epitope binding molecules that each recognizes a different 3 to 5 amino acid polypeptide epitope and one or more a small epitope binding molecules that each recognizes a different carbohydrate epitope, and the binding reagent as a whole recognizes a specific glycoprotein.

In some embodiments, a binding reagent contains 2, 3, or 4 small epitope binding molecules. The small epitope binding molecules may be linked covalently, for example, with a chemical cross-linking reagent, or noncovalently, for example, by interaction between members of a binding pair, such as, for example, avidin or streptavidin and biotin.

In some embodiments, a binding reagent comprises small epitope antibodies or epitope binding fragments thereof. A binding reagent comprising small epitope antibodies or epitope binding fragments thereof may be produced by recombinant expression as a polypeptide with a peptide linker between antibodies or epitope binding fragments, or via chemical crosslinking or linkage via binding between members of a binding pair.

The invention also provides a population of binding reagents, comprising a plurality of binding reagents each recognizing a different combination of small epitopes. In various embodiments, a population of binding reagents comprises any of about 2, 3, 5, 10, 20, 30, 40, 50, 75, 100, 125, 150, 200, 300, 400, 500, 1000, or more binding reagents. In some embodiments, a population of binding reagents comprises any of at least about 2, 3, 5, 10, 20, 30, 40, 50, 75, 90, 100, 125, 150, 200, 300, 400 or 500, with an upper limit of any of about 10, 20, 30, 40, 50, 75, 100, 125, 150, 200, 300, 400, 500, or 1000, binding reagents.

In some embodiments, a binding reagent of the invention contains one or more detectable labels. In some embodiments, the invention provides a population of binding reagents, comprising a plurality of binding reagents each with a different binding specificity (i.e., each recognizing and capable of binding to a different target molecule), and each comprising a detectable label. In one embodiment, each binding reagent in a population of binding reagents comprises a unique detectable label.

The invention also provides an array comprising a plurality of binding reagents immobilized on a support, such as a solid or semi-solid support, wherein each of binding reagents on the array recognizes a different combination of small epitopes. An “array” of binding reagents is an ordered spatial arrangement of a plurality of binding reagents on a physical substrate. Row and column arrangements are preferred due to the relative simplicity in making and assessing such arrangements. The spatial arrangement can, however, be essentially any form selected by the user, and preferably, but need not be, in a pattern.

The invention also comprises an affinity matrix comprising a binding reagent of the invention linked to a substrate, which may be used to enrich or purify a molecule of interest from a sample. A binding reagent as described herein is bound to a substrate to form an affinity matrix. The matrix is contacted by a solution which includes the binding partner of the binding reagent (i.e., a molecule of interest to which the binding reagent binds), allowing formation of a substrate-bound affinity complex. Formation of the affinity complex may occur in a column. Alternatively, affinity complexes may be formed in solution, with the binding reagent linked to a solid particle such as sepharose or agarose, with the complexes then isolated by centrifugation. To recover the molecule of interest, the affinity complex is destabilized, e.g., by exposure to buffers of very high ionic strength or high or low pH. In one embodiment, the affinity matrix comprises a binding reagent comprising small epitope antibodies, and the molecule of interest to be enriched or purified with the affinity matrix comprises is a polypeptide.

An affinity matrix comprises a binding reagent covalently attached to or associated with a matrix material. Non-limiting examples of matrix materials include solids, gels, pastes, membranes, slurries, or liquids. Suitable matrix materials include, but are not limited to, glass beads, controlled pore glass, magnetic beads, various membranes or rigid various polymeric resins such as polystyrene, polystyrene/latex, and other organic and inorganic polymers, both natural and synthetic. Illustrative polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, and nitrocellulose. Other materials that can be employed, include paper, glass, minerals (e.g. quartz), ceramics, metals, metalloids, plastics, cellulose, semiconductive materials, or cements. In addition, substances that form gels, such as proteins (e.g., gelatins), lipopolysaccharides, silicates, agarose, and polyacrylamides can be used. Polymers that form several aqueous phases, including, but not limited to, dextrans, polyalkylene glycols or surfactants, such as phospholipids, or long chain (12-24 carbon atoms) alkyl ammonium salts are also suitable.

The matrix material can take any of a number of morphologies. These include, but are not limited to solid or porous beads or other particles, solid surfaces (e.g. array substrates), columns, capillaries, or wells. In some embodiments, a plurality of different materials can be employed to form the affinity matrix, e.g., as laminates, to obtain various properties. For example, protein coatings, such as gelatin can be used to avoid nonspecific binding, simplify covalent conjugation, and/or enhance signal detection.

If covalent binding between the binding reagent and the matrix material is desired, the surface can be polyfunctional or can be capable of being polyfunctionalized. Functional groups that can be present on the surface and used for linking include, but are not limited to, carboxylic acids, aldehydes, amino groups, cyano groups, ethylenic groups, hydroxyl groups, mercapto groups, and haloacetyl groups (i.e., iodoacetyl groups).

In some embodiments, matrix materials include resins, such as for example synthetic resins (e.g. cross-linked polystyrene, divinyl benzene, etc.), and cross-linked polysaccharides (e.g. cellulose, dextran (sephadex), agarose (sepharose), and the like). In some embodiments, the matrix material includes reactive groups capable of forming a covalent link with a binding reagent. In one embodiment, the matrix material includes a glyoxal activated agarose. In another embodiment, the matrix material includes a sulfhydryl reactive group. In a still further embodiment, the matrix material is activated with cyanogen bromide. In other embodiments, a binding reagent is attached to an agarose resin by the use of a cross-linking reagent. Such reagents are well known to those of skill in the art and include, but are not limited to carbodiimides, maleimides, succinimides, and reactive disulfides. In other embodiments, a binding molecule is joined to a matrix material via a linker. Suitable linkers include, but are not limited to straight or branched-chain carbon linkers, heterocyclic carbon linkers, peptide linkers, and carbohydrates

An affinity matrix of the invention can take any convenient form. In certain embodiments, the affinity matrix is packed into a column, a mini-column, or a capillary or microcapillary (e.g., in a “lab on a chip” application), or a capillary electrophoresis tube. In some embodiments, the affinity matrix is suspended in one phase of a multiphase solution. In such embodiments, the affinity matrix thus acts to partition the tagged molecule into that particular phase of the multiphase (e.g., two-phase) system. Such multi-phase purification systems are well suited to large volume/high throughput applications.

In some embodiments, the affinity matrix comprises the walls of a vessel or the walls of a well (e.g., in a microtiter plate). In other embodiments, the affinity matrix comprises one or more porous or non-porous membranes or various gels or hydrogels. In one embodiment, the affinity matrix takes the form of a gel, such as for example a slab gel or a tube gel.

In another example of affinity purification, the binding reagent comprises small epitope antibodies and immunocomplex formation may be exploited to purify the molecule of interest by immunoprecipitation. Complexes comprising the binding reagent and the molecule of interest may be aggregated and precipitated following formation, followed by release of the antigen (i.e., the protein of interest) from the immunocomplex as described above.

### Small Epitope Antibodies

In some embodiments, the plurality of small epitope binding molecules in a binding reagent of the invention comprises one or more small epitope antibodies or epitope binding fragments thereof. In some embodiments, a binding reagent of the invention comprises 2, 3, or 4 small epitope antibodies or epitope binding fragments thereof.

As used herein, a “small epitope antibody” is an antibody that binds (generally specifically binds) a small peptide epitope. By virtue of the epitope specificity, an individual small epitope antibody generally recognizes a multiplicity of proteins that comprise the small epitope to which the antibody binds. Small epitope antibodies are described, for example, in U.S. Pat. No. 7,252,954 and in co-pending U.S. patent application Ser. No. 10/921,380 (publication no. 2005/0131219), and in PCT Publication Nos. WO 04/035742 and WO 05/019831. Small epitope antibodies and methods of making small epitope antibodies are further discussed herein.

A small epitope antibody or epitope binding fragment thereof encompasses monoclonal antibodies, polyclonal antibodies, antibody fragments (e.g., Fab, Fab′, F(ab′)₂, Fv, Fc, etc.), chimeric antibodies, single chain (ScFv), mutants thereof, fusion proteins comprising an antibody portion, and any other modified configuration of the immunoglobulin molecule that comprises a small epitope recognition site as described herein. A small epitope antibody or epitope binding fragment thereof may be of murine, rat, human, or any other origin (including humanized antibodies). Small epitope antibodies may be produced by a number of methods known in the art, including, for example, production by a hybridoma, recombinant production, or chemical synthesis.

Generally, a small epitope antibody binds a short, linear peptide epitope of 3, 4, or 5 sequential (consecutive) amino acids. Alternatively, in some embodiments, a small epitope antibody binds a discontinuous amino acid sequence within a polypeptide. In some embodiments, a small epitope antibody binds an epitope consisting of or consisting essentially of any of about 2, 3, 4, 5, 6, 7, 8, 9, or 10 amino acids. In some embodiments, a small epitope antibody binds an epitope consisting of or consisting essentially of 2 to 10, 3 to 8, or 3 to 5 amino acids. In one embodiment, a plurality of small epitope antibodies in a protein binding reagent of the invention binds epitopes of the same number of amino acids. In other embodiments, the plurality of small epitope antibodies binds epitopes of a mixture of different numbers of amino acids. In any of the embodiments described herein, an epitope may be a sequential or discontinuous sequence within a polypeptide, as described below.

In some embodiments, a small epitope antibody binds an epitope consisting of or consisting essentially of 3 sequential amino acids (termed a 3 mer), four sequential amino acids (termed a 4 mer), or five sequential amino acids (termed a 5 mer). In other embodiments, a small epitope antibody binds a small “discontinuous” or “degenerate” linear peptide sequence, such as the linear peptide sequence YCxC, wherein x represents any of the 20 natural amino acids (a degenerate linear sequence). In other embodiments, a small epitope antibody binds a non-sequential (discontinuous) sequence within a polypeptide based on conformational proximity of amino acids within the polypeptide to form the epitope (for example, a conformational epitope formed by proximity of amino acid residues due to secondary structure within a folded polypeptide). In still other embodiments, a small epitope antibody may bind an epitope consisting of an amino acid sequence that is predicted to be antigenic, using methods well known in the art for predicting antigenicity. Antibodies that bind small linear peptide epitopes have been previously described, as shown in Table 2, below. In some embodiments, the same antibody may bind a sequential sequence on one or more proteins and a discontinuous sequence on one or more proteins.

Small epitope antibodies generally recognize a multiplicity of proteins that comprise the small epitope to which the antibody binds. In some embodiments, the small epitope antibody binds to an epitope present one or more times in about any of 0.1%, 0.5%, 1, 2%, 3%, 4%, 5%, 10%, or more of proteins in a sample. In still other embodiments, the small epitope antibody binds to an epitope present one or more times in about 0.1% to 1% of proteins in a sample. In still other embodiments, the small epitope antibody binds to an epitope present one or more times in approximately 1-5% of proteins in a sample. In still other embodiments, the small epitope antibody binds to an epitope present one or more times in about 0.1% to 1% of proteins in a sample, wherein the small antibody epitope binds to a linear peptide epitope consisting of or consisting essentially of 3 amino acids, 4 amino acids or 5 amino acids. In still other embodiments, the small epitope antibody binds to an epitope present one or more times in about 1-5% of proteins in a sample, wherein the small antibody epitope binds to a linear peptide epitope consisting of or consisting essentially of 3 amino acids, 4 amino acids or 5 amino acids. In still other embodiments, the small epitope antibody binds to an epitope present one or more times in about 5-7% or about 5-10% of proteins in a sample, wherein the small antibody epitope binds to a linear peptide epitope consisting or consisting essentially of 3 amino acids, 4 amino acids or 5 amino acids. Methods for empirically assessing frequency of an epitope in a sample include: assessment using biochemical approaches, such as binding of an antibody followed by analysis using, for example, 2D gels or mass spectrometry, and sequence based analysis, using, for example, amino acid or nucleic acid sequence databases such as GenBank and SwissProt. Suitable databases are further described herein.

In some embodiments, a small epitope antibody binds its cognate epitope with an affinity of a binding reaction of at least about 10⁻⁷ M, at least about 10⁻⁸ M, or at least about 10⁻⁹ M, or lower. Binding affinity may be measured by well-known methods in the art, including, for example, by surface plasmon resonance (Malmborg and Borrebaeck (1995) J. Immunol. Methods 183(1):7-13; Lofas and Johnsson (1990) J. Chem. Soc. Chem. Commun. 1526. In some embodiments, a binding interaction will discriminate over adventitious binding interactions in the reaction by at least two-fold, at least five-fold, at least 10- to at least 100-fold or more.

Antibodies that bind small linear peptide epitopes have been previously described, as shown in Table 1.

TABLE 1
Published short antibody epitope sequences

| Epitope Seq | Source protein | Antibody | Reference |
|---|---|---|---|
| NKS | Opa of N. | U623, U506 | Malorny, B., et al. |
| | meningitidis | | (1998) J Bacteriol |
| | | | 180(5): 1323-30. |
| NRQD | Opa of N. | O521 | Id. |
| | meningitides | | |
| TTFL | Opa of N. | AB419 | Id. |
| | meningitides | | |
| NIP | Opa of N. | W320/15, | Id. |
| | meningitides | W124 | |
| GAT | Opa of N. | P515 | Id. |
| | meningitides | | |
| EQP | MB of U. | 3B1.5 | Zheng, X., et al., |
| | urealyticum | | (1996) Clin Diagn |
| | | | Lab Immunol 3(6): |
| | | | 774-8. |
| WQDE | Porcine ZP3 beta | mAb-30 | Afzalpurkar, A. et |
| | | | al. (1997) Am J |
| | | | Reprod Immunol |
| | | | 38(1): 26-32. |
| GPGR | Gp120 of HIV-1 | 9x mAbs | Akerblom, L., et |
| | | | al. (1990) Aids |
| | | | 4(10): 953-60. |
| D(A/S)F* | Phosphofructokinase-1 | alpha-F3 | Hollborn, M., et |
| | | | al. (1999) J Mol |
| | | | Recognit 12(1): |
| | | | 33-7. |
| (D/S)GY(A/G)** | Crotoxin | A-56.36 | Demangel, C., et |
| | | | al. (2000) Eur J |
| | | | Biochem 267(8): |
| | | | 2345-53 |

*DAF and DSF.
**Refers to DGYA, DGYG, SGYA and SGYG.

Methods of making small epitope antibodies are known in the art. Small epitope antibodies (e.g., human, humanized, mouse, chimeric) may be made by using immunogens which express one or more small peptide epitopes, such as a small linear peptide epitope consisting of or consisting essentially of 3, 4, or 5 amino acids.

Immunogens may be produced, for example, by chemical synthesis. Methods for synthesizing polypeptides are well known in the art. In some embodiments, the polypeptide immunogen is synthesized with a terminal cysteine to facilitate coupling to either KLH or BSA, as is known in the art. The terminal cysteine can be incorporated at the amino terminus of the polypeptide (which may minimize steric effects during immunization and screening), or at the carboxy terminus. In other embodiments, the polypeptide immunogen is synthesized as a multiple antigen polypeptide, or MAP.

The route and schedule of immunization of the host animal are generally in keeping with established and conventional techniques for antibody stimulation and production, as further described herein. General techniques for production of human and mouse antibodies are known in the art and are described herein. Typically, the host animal is inoculated intraperitoneally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W. et al., (1982) In Vitro, 18:377-381. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the small epitope antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas or progeny cells of the parent hybridomas that produce small epitope antibodies (such as monoclonal antibodies) may be used as source of antibodies or derivatives thereof, or a portion thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a human or other species of small epitope receptor, or a fragment of the human or other species of small epitope receptor, or a human or other species of small epitope receptor or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaradehyde, succinic anhydride, SOCl2, or R1N═C═NR, where R and R1 are different alkyl groups, can yield antibodies (e.g., monoclonal antibodies).

If desired, the small epitope antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. It may be desirable to genetically manipulate the antibody sequence to obtain greater affinity to the small epitope and/or greater and/or altered specificity to the small epitope. It will be apparent to one of skill in the art that one or more polynucleotide changes can be made to the small epitope antibody and still maintain its binding ability to the small epitope.

Antibodies may be made recombinantly and expressed using any method known in the art. In another alternative, antibodies may be made recombinantly by phage display technology. See, for example, U.S. Pat. Nos. 5,565,332; 5,580,717; 5,733,743 and 6,265,150; Winter et al. (1994) Annu. Rev. Immunol. 12:433-455; Bradbury and Marks (2004) J. Immunological Methods 290:29-49. In other embodiments, antibodies may be produced by yeast display (see, for example, Feldhaus and Siegel (2004) J. Immunological Methods 290:69-80) or by ribosome display (see, for example, Roberts and Szostak (1997) Proc Natl Acad Sci 94:12297-12302; Schaffitzel et al. (1999) J. Immunolical Methods 231:119-135; Lipovsek and Plijckthun (2004) J. Immunological Methods 290:51-67; World Wide Web at discerna.co.uk/research.htm).

Antibodies may be made recombinantly by first isolating the antibodies made from host animals, obtaining the gene sequence, and using the gene sequence to express the antibody recombinantly in host cells (e.g., CHO cells). Another method that may be employed is to express the antibody sequence in plants (e.g., tobacco), transgenic milk, or in other organisms. Methods for expressing antibodies recombinantly in plants or milk have been disclosed. See, for example, Peeters et al. (2001) Vaccine 19:2756; Lonberg, N. and D. Huszar (1995) Int. Rev. Immunol 13:65; and Pollock et al. (1999) J Immunol Methods 231:147. Methods for making derivatives of antibodies, e.g., humanized, single chain, etc. are known in the art.

Immunoassays and flow cytometry sorting techniques such as fluorescence activated cell sorting (FACS) can also be employed to isolate antibodies that are specific for the desired small epitope.

DNA encoding small epitope antibodies may be isolated and sequenced, as is known in the art. Generally, the monoclonal antibody is readily isolated and sequenced using conventional procedures (e.g., by using oligonucleotide probes that are capable of binding specifically to genes encoding the heavy and light chains of the monoclonal antibodies). The hybridoma cells serve as a preferred source of such cDNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morrison et al. (1984) Proc. Nat. Acad. Sci. 81: 6851, or by covalently joining to the immunoglobulin coding sequence all or part of the coding sequence for a non-immunoglobulin polypeptide. In that manner, “chimeric” or “hybrid” antibodies are prepared that have the binding specificity of a small epitope antibody (such as a monoclonal antibody) herein.

Small epitope antibodies may be characterized using methods well-known in the art, some of which are described in the Examples. For example, one method is to identify the epitope to which it binds, including solving the crystal structure of an antibody-antigen complex, competition assays, gene fragment expression assays, and synthetic polypeptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which a small epitope antibody binds. Epitope mapping is commercially available from various sources, for example, Pepscan Systems (Edelhertweg 15, 8219 PH Lelystad, The Netherlands). Polypeptides of varying lengths (e.g., at least 4-6 amino acids long) can be isolated or synthesized (e.g., recombinantly) and used for binding assays with an anti-small epitope antibody. In another example, the epitope to which the small epitope antibody binds can be determined in a systematic screening by using overlapping polypeptides derived from the small epitope extracellular sequence and determining binding by the small epitope antibody. Certain epitopes can also be identified by using large libraries of random polypeptide sequences displayed on the surface of phage particles (phage libraries), as is well known in the art.

Yet another method which can be used to characterize an anti-small epitope antibody is to use competition assays with other antibodies known to bind to the same antigen, i.e., to determine if the anti-small epitope antibody binds to the same epitope as other antibodies. Competition assays are well known to those of skill in the art.

### Detectable Labels

In some embodiments, a binding reagent of the invention comprises a detectable label. “Label” as used herein refers to a composition capable of producing a detectable signal. A label is any composition detectable by spectroscopic, photochemical, biochemical, immunochemical, electrical, optical, chemical, or any other means by which the label may be detected quantitatively and/or qualitatively. The term “label” refers to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. A label may be attached via a linker. Examples of labels include, but are not limited to, biotin, avidin, streptavidin, digoxigenin, fluorophors (e.g., fluoroescein, acetylaminofluorene), chromophors, magnetically responsive compounds, antibody epitope-containing compounds, haptens, radiolabels (e.g., ¹²⁵I, ³²P, ³³P, ³H, ¹⁴C, ³⁵S), detectable isotopes (e.g., ²H), chemiluminescent labels, bioluminescent labels, enzymes, or magnetic labels such as magnetic beads.

The label may also be a compound that is a member of a binding pair, one member of which bears a detectable moiety or physical property. The terms “binding partner,” “member of a binding pair,” or “cognate ligand” refer to molecules that specifically bind other molecules to form a binding complex, such as, for example, antibody/antigen, lectin/carbohydrate, nucleic acid/nucleic acid, receptor/receptor ligand, avidin/biotin or streptavidin, etc. The label may also be a moiety that is suitable for detection by mass spectrometry.

A label may be a member of a signal producing system that acts in concert with one or more additional members of the same system to provide a detectable signal. Illustrative of such labels are members of a specific binding pair, such as a ligand, e.g., biotin, fluorescein, digoxigenin, antigen, polyvalent cation, or a chelator group, where the member specifically binds to one or more additional members of the signal producing system, wherein the additional member(s) provide a detectable signal either directly or indirectly, e.g., antibody conjugated to a fluorescent moiety or an enzymatic moiety capable of converting a substrate to a chromogenic product, e.g., alkaline phosphatase conjugated antibody. The label is subsequently detected by colorimetry or chemiluminescence, for example as described by Coutlee, et al. (1989) J. Clin. Microbiol. 27:1002-1007. In one embodiment, bound alkaline phosphatase is detected by chemiluminescence with a reagent such as a Lumi-Phos™ luminometer (source Scientific Systems, Inc., Garden Grove, Calif.).

Flourescent labels include coumarin and its derivatives (e.g., 7-amino-4-methylcoumarin, aminocoumarin), bodipy dyes (e.g., Bodipy FL, cascade blue), fluorescein and its derivatives (e.g., fluorescein isothiocyanate, Oregon green), rhodamine dyes (e.g., Texas red, tetramethylrhodamine, eosins and erythrosins), cyanine dyes (e.g., Cy3 and C65), macrocyclic chelates of lanthanide ions (e.g., quantum Dye™), and fluorescent energy transfer dyes (e.g., thiazole orange-ethidium heterodimer, TOTAB, etc.).

In some embodiments, the invention provides a plurality of binding reagents, each comprising a “unique detectable label” or “coded label” which is uniquely coded such that it may be distinguished from other unique detectable labels attached to other binding reagents. Examples of unique detectable labels for use in accordance with the methods of the invention include, but are not limited to, color-coded microspheres of known fluorescent light intensities (e.g., produced by Luminex (World Wide Web at luminexcorp.com); microspheres containing quantum dot nanocrystals, for example, containing different ratios and combinations of quantum dot colors (e.g., Qbead™ microspheres produced by Quantum Dot Corporation, World Wide Web at qdots.com); glass coated metal nanoparticles (e.g., SERS nanotags produced by Nanoplex Technologies, Inc., worldwide web at nanoplextech.com); barcode materials (e.g., sub-micron sized striped metallic rods (such as Nanobarcodes® Particles produced by Nanoplex Technologies, Inc.), encoded microparticles with colored bar codes (such as CellCard™ produced by Vitra Bioscience, World Wide Web at vitrabio.com), glass microparticles with digital holographic code images (such as CyVera microbeads produced by Illumina, World Wide Web at illumina.com); chemiluminescent dyes, combinations of dye compounds; and beads of detectably different sizes.

Methods of Making Binding Reagents

The invention also provides methods for making a binding reagent comprising a plurality of linked small epitope binding molecules, wherein each small epitope binding molecule recognizes a different small epitope.

In one embodiment, the method comprises covalently linking the plurality of small epitope binding molecules. In some embodiments, 2, 3, or 4 small epitope binding molecules, such as, for example, small epitope antibodies, are covalently linked. In some embodiments, small epitope protein molecules are covalently linked via a chemical cross-linking reagent. In some embodiments, small epitope binding molecules are covalently linked via a linker, such as, for example, a peptide or carbohydrate linker.

In another embodiment, the method comprises linking the plurality of small epitope binding molecules, such as, for example, small epitope antibodies, noncovalently. In some embodiments, 2, 3, or 4 small epitope binding molecules are noncovalently linked. In some embodiments, small epitope binding molecules are noncovalently linked via interaction between members of a binding pair.

In another embodiment, the method comprises linking a plurality of polypeptide small epitope binding molecules (e.g., small epitope antibodies or small epitope binding fragments thereof) by recombinant expression. In one embodiment, expression is effected from a vector that comprises sequences encoding peptide linkers between sequences encoding small epitope antibodies or small epitope binding fragments thereof. In another embodiment, the small epitope antibodies or epitope binding fragments thereof are expressed from a vector comprising a contiguous sequence encoding the small epitope antibodies or epitope binding fragments thereof without peptide linkers in between.

In some embodiments, the method further comprises attaching one or more detectable label to the binding reagent. In one embodiment, one or more labels are attached to the binding reagent after linkage of the small epitope binding molecules to form the binding reagent. In another embodiment, one or more small epitope binding molecules comprises a detectable label prior to incorporation into the binding reagent.

Cross-Linking Reagents and Linkers

In some embodiments, small epitope binding molecules are linked via a chemical cross-linking reagent to produce a binding reagent. Cross-linking reagents are available, for example, from Pierce Chemical (World Wide Web at piercenet.com). Such reagents are well known to those of skill in the art and include, but are not limited to, carbodiimides, maleimides, succinimides, and reactive disulfides. In some embodiments, a heterobifunctional chemical cross-linking reagent may be used for linking with two reactive groups that are capable of reacting with and forming links, or bridges, between the side chains of certain amino acids, between amino acids and carboxylic acid groups, or via carbohydrate moieties. For example, heterobifunctional cross-linking reagents may be used that contain either a cleavable disulfide bond group or a maleimido group.

In some embodiments, small epitope binding molecules are linked via a linker. Suitable linkers include, but are not limited to straight or branched-chain carbon linkers, heterocyclic carbon linkers, peptide linkers, and carbohydrates.

Binding Pairs

In some embodiments, small epitope binding molecules are linked via a chemical cross-linking via interaction between members of a binding pair to produce a binding reagent. Such binding pairs are well known to those of skill in the art and include, but are not limited to, antibody/antigen, lectin/carbohydrate, nucleic acid/nucleic acid, receptor/receptor ligand, avidin/biotin, streptavidin/biotin, hydroxysuccinimide/borate (e.g., phenyldiboronic acid (PDBA)/salicylhydroxamic acid (SHA); Springer et al. (2003) Journal of Biomolecular Techniques 14:183-90), enzyme/pseudosubstrate, HISx/metal complex, etc.

Recombinant Expression

In some embodiments, a binding molecule of the invention is produced by a method that comprises recombinant expression of polypeptide small epitope binding molecules, for example, small epitope antibodies, or alternative scaffolds such as anticalins or T cell receptors. In some embodiments, the method comprises expression from a polynucleotide vector that encodes small epitope antibody or alternative scaffold sequences with intervening sequences encoding a peptide linker between antibody sequences. Expression from the vector produces a polypeptide comprising small epitope antibodies or alternative scaffolds linked via peptide linker(s). In other embodiments, the method comprises expression from a polynucleotide vector that contiguously encodes small epitope antibody of alternative scaffold sequences without intervening peptide linker sequences.

Methods for recombinant expression are routine and are well known to those of skill in the art. For instance, a polynucleotide encoding polypeptide sequences such as heavy or light chains of small epitope antibodies, with or without intervening sequences encoding peptide linker(s), can be cloned into a suitable expression vector (which contains control sequences for transcription, such as a promoter). The expression vector is in turn introduced into a host cell. The host cell is grown under suitable conditions such that the polynucleotide is transcribed and translated into a protein. Antibody heavy and light chains may be produced separately, and then combined by disulfide bond rearrangement. Alternatively, vectors with separate polynucleotides encoding heavy and light chains, or a vector with a single polynucleotide encoding both chains as separate transcripts, may be transfected into a single host cell which may then produce and assemble the entire molecule. The polypeptide thus produced in the host cell can be purified using standard techniques in the art. A polynucleotide encoding small epitope antibody sequences for use in the production of a binding reagent by any of these methods can be obtained from a hybridoma producing the antibody, or may be produced synthetically or recombinantly from a known DNA sequence that encodes antibody polypeptide sequences.

The invention also provides expression vectors that comprise polynucleotide sequences encoding two or more small epitope antibody or alternative scaffold polypeptide sequences with or without an intervening polynucleotide sequence encoding a peptide linker in between sequences encoding the small epitope antibodies or alternative scaffolds, wherein the small epitope antibodies or alternative scaffolds encoded by the polynucleotide sequences of the vector each recognize a different small epitope. The design of a protein expression vector can depend on such factors as the choice of the host cell to be transfected and/or particular polypeptide(s) to be expressed. It will be appreciated that polynucleotide sequences can be operably linked to constitutive promoters for high level, continuous expression. Alternatively, inducible promoters can be utilized. When used in mammalian cells, a recombinant expression vector's control functions are often provided by a promoter, often of viral origin. Promoters include, but are not limited to CMV immediate early, HSV thymidine kinase, early and late SV40, LTRs from retrovirus, and mouse metallothionein-I. Use of appropriate regulatory elements can allow for high level expression of the polypeptide(s) in a variety of host cells.

In some embodiments, a recombinant expression vector for production of a binding reagent is a plasmid or cosmid. In other embodiments, the expression vector is a virus, or portion thereof, that allows for expression of a nucleic acid introduced into the viral nucleic acid. For example, replication defective retroviruses, adenoviruses and adeno-associated viruses can be used.

Expression vectors may be derived from bacteriophage, including all DNA and RNA phage (e.g., cosmids), or viral vectors derived from all eukaryotic viruses, such as baculoviruses and retroviruses, adenoviruses and adeno-associated viruses, Herpes viruses, Vaccinia viruses and all single-stranded, double-stranded, and partially double-stranded DNA viruses, all positive and negative stranded RNA viruses, and replication defective retroviruses. Another example of an expression vector is a yeast artificial chromosome (YAC), which contains both a centromere and two telomeres, allowing YACs to replicate as small linear chromosomes. A number of suitable expression systems are commercially available and can be modified to produce the vectors of this invention. Illustrative expression systems include, but are not limited to baculovirus expression vectors (see, e.g., O'Reilly et al. (1992) Baculovirus Expression Vectors. A Laboratory Manual, Stockton Press) for expression in insect (e.g. SF9) cells, a wide variety of expression vectors for mammalian cells (see, e.g., pCMV-Script® Vector, pCMV-Tagl, from Stratagene), vectors for yeast (see, e.g., pYepSec1, Baldari et al. (1987) EMBO J. 6: 229-234, pMFa (Kurjan and Herskowitz, (1982) Cell 30: 933-943), pJRY88 (Schultz et al., (1987) Gene 54:113-123), pYES2 (Invitrogen Corporation, San Diego, Calif.), and the like), prokaryotic vectors (see, e.g., arabinose-regulated promoter (Invitrogen pBAD Vector), T7 Expression Systems (Novagen, Promega, Stratagene), Trc/Tac Promoter Systems (Clontech, Invitrogen, Kodak, Life Technologies, MBI Fermentas, New England BioLabs, Pharmacia Biotech, Promega), PL Promoters (Invitrogen pLEX and pTrxFus Vectors), Lambda PR Promoter (Pharmacia pRIT2T Vector), Phage T5 Promoter (QIAGEN), tetA Promoter (Biometra pASK75 Vector), and the like.

The binding reagents of the invention can be expressed in a host cell. In one embodiment, the invention provides a host cell comprising an expression vector encoding polypeptide sequences of a binding reagent, i.e., polypeptides sequences of small epitope binding molecules, such as small epitope antibodies, or alternative scaffolds such as anticalins or T cell receptors, as described above. As used herein, the term “host cell” is intended to include any cell or cell line into which a recombinant expression vector for production of a binding reagent, as described above, may be transfected for expression. Host cells include progeny of a single host cell, and the progeny may not necessarily be completely identical (in morphology or in total genomic DNA complement) to the original parent cell due to natural, accidental, or deliberate mutation. A host cell includes cells transfected or transformed in vivo with an expression vector as described above.

Suitable host cells include, but are not limited to, to algal cells, bacterial cells (e.g. E. coli), yeast cells (e.g., S. cerevisiae, S. pombe, P. pastoris, K. lactis, H. polymorpha, see, e.g., Fleer (1992) Curr. Opin. Biotech. 3(5): 486-496), fungal cells, plant cells (e.g. Arabidopsis), invertebrate cells (e.g. insect cells such as SF9 cells, and the like), and vertebrate cells including mammalian cells. Non-limiting examples of mammalian cell lines which can be used include CHO cells (Urlaub and Chasin (1980) Proc. Natl. Acad. Sci. USA 77: 4216-4220), 293 cells (Graham et al. (1977) J. Gen. Virol. 36: 59), or myeloma cells like (e.g., SP2 or NSO, see Galfre and Milstein (1981) Meth. Enzymol. 73(B):3-46). In one embodiment, the expression system includes a baculovirus vector expressed in an insect host cell.

An expression vector encoding a binding reagent can be transfected into a host cell using standard techniques. “Transfection” or “transformation” refers to the insertion of an exogenous polynucleotide into a host cell. The exogenous polynucleotide may be maintained as a non-integrated vector, for example, a plasmid, or alternatively, may be integrated into the host cell genome. The term “transfecting” or “transfection” is intended to encompass all conventional techniques for introducing nucleic acid into host cells. Examples of transfection techniques include, but are not limited to, calcium phosphate precipitation, DEAE-dextran-mediated transfection, lipofection, electroporation and microinjection. Suitable methods for transfecting host cells can be found in Sambrook et al. (1989) Molecular Cloning. A Laboratory Manual, 2^{nd} Edition, Cold Spring Harbor Laboratory press, and other laboratory textbooks. Nucleic acid can also be transferred into cells via a delivery mechanism suitable for introduction of nucleic acid into cells in vivo, such as via a retroviral vector (see e.g., Ferry et al. (1991) Proc. Natl. Acad. Sci., USA, 88: 8377-8381; and Kay et al. (1992) Human Gene Therapy 3: 641-647), an adenoviral vector (see, e.g., Rosenfeld (1992) Cell 68: 143-155; and Herz and Gerard (1993) Proc. Natl. Acad. Sci., USA, 90:2812-2816), receptor-mediated DNA uptake (see e.g., Wu, and Wu (1988) J. Biol. Chem. 263: 14621; Wilson et al. (1992) J. Biol. Chem. 267: 963-967; and U.S. Pat. No. 5,166,320), direct injection of DNA (see, e.g., Acsadi et al. (1991) Nature 332: 815-818; and Wolff et al. (1990) Science 247:1465-1468) or particle bombardment (biolistics) (see e.g., Cheng et al. (1993) Proc. Natl. Acad. Sci., USA, 90:4455-4459; and Zelenin et al. (1993) FEBS Letts. 315: 29-32).

Certain vectors integrate into host cells at a low frequency. In order to identify these integrants, in some embodiments a gene that contains a selectable marker (e.g., drug resistance) is introduced into the host cells along with the nucleic acid of interest. Examples of selectable markers include those which confer resistance to certain drugs, such as G418 and hygromycin. Selectable markers can be introduced on a separate vector from the nucleic acid of interest or on the same vector. Transfected host cells can then be identified by selecting for cells using the selectable marker. For example, if the selectable marker encodes a gene conferring neomycin resistance, host cells which have taken up nucleic acid can be identified by their growth in the presence of G418. Cells that have incorporated the selectable marker gene will survive, while the other cells die.

Once expressed, a binding reagent can be purified according to standard procedures of the art, including, but not limited to affinity purification, ammonium sulfate precipitation, ion exchange chromatography, or gel electrophoresis (see generally, R. Scopes, (1982) Protein Purification, Springer-Verlag, N.Y.; Deutscher (1990) Methods in Enzymology Vol. 182: Guide to Protein Purification., Academic Press, Inc. N.Y.).

Methods of Use

Binding reagents of the invention may be used in any application for which an antibody molecule is conventionally used. For example, binding reagents of the invention may be used in methods for purifying or separating a molecule of interest, such as a protein, from a mixture or sample. Binding reagents of the invention may also be used for characterizing (for example, detecting (presence or absence) and/or quantifying) a molecule of interest, such as a protein. In some embodiment, a binding reagent is used in a diagnostic application to detect presence, absence, or amount of a metabolite, biomarker, environmental toxin, etc. Binding reagents of the invention may also be used as therapeutic agents or for delivery of a therapeutic agent to an in vivo site of action.

In some embodiments, a binding reagent of the invention is used for purification or separation of a molecule of interest from a mixture. For example, binding reagents of the invention may be used in affinity purification procedures, such as affinity chromatography or precipitation or removal of a binding reagent-molecule of interest complex from a mixture.

In one embodiment, a binding reagent attached to a substrate to form an affinity matrix. The affinity matrix may be contacted with a mixture containing the molecule of interest. The molecule of interest may bind to the affinity matrix and unbound molecules in the mixture may be eluted or otherwise removed from contact with the affinity matrix. In one embodiment, the molecule of interest is a protein. In one embodiment, the binding reagent comprises small epitope antibodies.

In another embodiment, a mixture containing a molecule of interest is contacted with a binding reagent that binds to the molecule of interest to form a binding reagent-molecule of interest complex. In one embodiment, the complex is removed from other components of the mixture by precipitation. In one embodiment, the binding reagent comprises small epitope antibodies and the complex is removed from the mixture by immunoprecipitation.

In one embodiment, the molecule of interest is a protein. In one embodiment, the binding reagent comprises small epitope antibodies.

In some embodiments, binding reagents of the invention are used for qualitative (determination of presence or absence, i.e., detection) and/or quantitative (determination of amount) of a molecule of interest in a sample or mixture.

In one embodiment, binding reagent that binds to a molecule is attached or associated with a substrate and contacted with a sample. Unbound molecules are removed, for example, by washing the substrate, centrifugation, etc. Molecules of interest that bind to the binding reagent on the substrate are detected and/or quantitated. Detection may be via detection of a detectable characteristic or label attached to the molecule of interest, or via binding and detection of one or more additional reagents to the molecule of interest, for example, a binding reagent as described herein or an antibody that binds to the molecule of interest, or a detectable member of a binding pair that binds to the molecule of interest, which has been labeled with the other member of the binding pair. In one embodiment, the molecule of interest is a protein. In one embodiment, the binding reagent comprises small epitope antibodies.

In another embodiment, a sample is attached to or associated with a substrate, and the substrate is contacted with a binding reagent as described herein. Unbound binding reagent is removed, for example, by washing, centrifugation, etc. The molecule of that was present in the sample is detected and/or quantitated by detection and/or quantitation of bound binding reagent. Detection may be via detection of a detectable characteristic of the binding reagent or label attached to the binding reagent, or via binding and detection of one or more additional reagents to the binding reagent, for example, a second binding reagent as described herein or an antibody that binds to the binding reagent, or a detectable member of a binding pair that binds to the binding reagent, which has been labeled with the other member of the binding pair. In one embodiment, the molecule of interest is a protein. In one embodiment, the binding reagent comprises small epitope antibodies.

In some embodiments, a plurality of molecules of interest is characterized with a plurality of binding reagents, each of which recognizes a different molecule of interest. In one embodiment, each binding reagent in the plurality of binding reagents is labeled with a unique detectable label, and characterization of molecules of interest comprises detection and/or quantitation of binding of each of the binding reagents in the plurality of binding reagents. In one embodiment, molecules in a sample comprise a detectable label, such as, for example, biotin, biotin, avidin, streptavidin, fluorophors, an enzymatic label, or a radiolabel, the sample is contacted with a plurality of binding reagents each comprising a unique detectable label, examples of which are described above, and characterizing molecules of interest in the sample comprises detection of both the label attached to the molecules of interest and the unique detectable labels attached to each of the binding reagents. In some embodiments, a mixture or sample is contacted with any of at least about 2, 3, 4, 5, 10, 20, 30, 40, 50, 75, 100, 125, 150, 200, 300, 400, 500, or 1000 binding reagents. In some embodiments, a mixture or sample is contacted with any of at least about 2, 3, 4, 5, 10, 20, 30, 40, 50, 60, 70, 80, 90, 100, 125, 150, 200, 300, 400, or 500 binding reagents, with an upper limit of any of at least about 10, 20, 30, 45, 50, 75, 90, 100, 125, 150, 200, 300, 400, 500, or 1000 binding reagents. In some embodiments, the invention provides an array comprising a plurality of binding reagents immobilized on a support, wherein each of the binding reagents recognizes a different combination of small epitopes, the array is contacted with a sample or mixture, and molecules bound to each of the binding reagents on the array, if any, are detected. In some embodiments, molecules in a sample are detectably labeled and the labeled molecules bound to the array, if any, are detected. In one embodiment, the molecules of interest are proteins. In one embodiment, the binding reagent comprises small epitope antibodies.

In some embodiments, binding reagents are used to characterize a protein of interest, as described herein, and characterization includes analysis for the detection of any alterations in the protein, as compared to a reference protein which is identical (at least in part) to the protein sequence other than the sequence alteration. The sequence alterations may be sequence alterations present in the genomic sequence or may be sequence alterations which are not reflected in the genomic DNA sequences, for example, alterations due to post transcriptional alterations, and/or mRNA processing, including splice variants, and/or post-translational modifications, such as variation in amount of glycosylation, and protein degradation or by-products. Sequence alterations include mutations (such as deletion, substitution, insertion and/or transversion of one or more amino acid).

The invention also provides a method for identifying a binding reagent specific for a molecule of interest, comprising contacting the molecule of interest with a population of binding reagents each recognizing a different combination of small epitopes (i.e., a library of binding reagents), and detecting formation of binding reagent-molecule of interest complex(es), if any. In one embodiment, the method comprises contacting an array of binding reagents with the molecule of interest, and detecting the molecule of interest bound to binding reagents on the array.

In some embodiments, binding reagents of the invention are used as therapeutic agents. A binding reagent is administered to an individual to whom such administration is therapeutically beneficial. For example, a binding reagent may bind to a protein expressed in a tumor cell, thereby inhibiting tumor growth, or may bind to a cell surface receptor or ligand thereof, thereby activating or reducing receptor activity and/or signal transduction through the receptor. A binding reagent may be administered in a pharmaceutical composition, comprising one or more binding reagents as described herein and a pharmaceutically acceptable carrier.

In one embodiment, a binding reagent binds a phosphorylation site and an SH2 domain from the same or different protein order to keep the phosphorylation site accessible or inaccessible to maintain or prevent signaling. In one embodiment, a binding reagent is used to prevent inter- or intra-molecular binding events that may be necessary to initiate a signal cascade. In one embodiment, a binding reagent is used to tip the balance between the genes Bad, Bcl-X and Bim or Bax and Bcl-2 to control mitochondrial cell death and apoptosis. In one embodiment, a binding reagent binds a protease inhibitor to the protease to prevent its functioning in initiation of cell signaling, e.g., matrix metalloproteases and their inhibitors. In one embodiment, part of a binding reagent recognizes an epitope sequence common to different splice variants of a gene product and part of the binding reagent recognizes a sequence specific to only one splice variant.

In some embodiments, binding reagents of the invention are used a targeting agents for delivery of therapeutic substances in vivo, for example, a therapeutic radioisotope, a boron addend, an anti-pathogenic drug, a cytotoxic agent, or a chemotherapeutic agent, conjugated to the binding reagent. In one embodiment, an anti-tumor agent may be delivered by administration of a binding reagent that binds to a protein expressed in a tumor cell. A targeting may be administered as a pharmaceutical composition, comprising one or more binding reagents as described herein, conjugated to one or more therapeutic substances, and a pharmaceutically acceptable carrier.

In some embodiments, binding reagents of the invention are used as in vivo imaging agents.

Detection

In some embodiments, methods for characterization of molecules of interest comprise detection of a detectable label attached to or associated with a molecule of interest and/or a binding reagent that binds to the molecule of interest. Detection of a label may be direct or indirect. As used herein, “direct” detection refers to detection of a label that is attached to a molecule of interest or to a binding reagent that binds to the molecule (e.g., detection of a fluorescent label that is covalently attached to a molecule of interest or to a binding reagent that binds to the molecule of interest), and “indirect” refers to detection of a molecule bound to or associated with a molecule or moiety attached to a molecule of interest or to a binding reagent that binds to the molecule (e.g., detection of streptavidin binding to a biotin label covalently attached to a molecule of interest or to a binding reagent that binds to the molecule of interest).

In one embodiment, either a molecule of interest or a binding reagent comprises biotin and detection comprises binding of a fluorescent streptavidin label, and detection of fluorescence. Quantification may comprise assessment of the amount of label detected (e.g., strength of signal) with respect to one or more molecules in a sample, or with respect to a known standard.

In some embodiments, a molecule of interest to be detected is a protein. Identification of one or more proteins may comprise comparison of binding characteristics of a protein bound to a binding reagent of the invention with one or more characteristics of a known protein (e.g., binding affinity to the protein binding reagent, size, chemical, or physical characteristics.

Proteins (i.e., a protein of interest) may be labeled by incorporating a label at the C-terminus, N-terminus, and/or at one or more interior amino acid residues (i.e., amino acid residues that comprise a reactive nucleophilic moiety, for example, Lys, Arg, Cys). In some embodiments, proteins in a sample or protein containing mixture are differentially labeled with different labels at different positions in the polypeptide. In some embodiments, proteins are labeled and then cleaved, chemically or enzymatically, to produce polypeptide fragments, and only the labeled fragments are detected, thereby simplifying analysis. Incorporation of a label may also be used to separate labeled fragments from non-labeled fragments. For example, the N-termini of proteins could be labeled with biotin and then separated from non-labeled fragments after chemical or enzymatic cleavage of the polypeptides by binding to a reagent containing avidin or streptavidin, thus producing a protein mixture essentially containing only N-terminal fragments.

Sample

As noted in the definition and as used herein, “sample” encompasses a variety of sample types and/or origins, such as blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “sample” encompasses a clinical sample, and also includes cells in culture, cell supernatants, cell lysates, serum, plasma, biological fluid, and tissue samples. A sample can be from a microorganism, e.g., bacteria, yeasts, viruses, viroids, molds, fungi, plants, animals, including mammals such as humans. A sample may comprise a single cell or more than a single cell. Examples of a sample include blood, plasma, serum, urine, stool, cerebrospinal fluid, synovial fluid, amniotic fluid, saliva, lung lavage, semen, milk, nipple aspirate, prostatic fluid, mucous, cheek swabs, and/or tears.

These samples can be prepared by methods known in the art such as lysing, fractionation, purification, including affinity purification, FACS, laser capture microdissection (LCM) or isopycnic centrifugation. In some embodiments, subcellular fractionation methods are used to create enriched cellular or subcellular fractions, such as subcellular organelles including nuclei, mitochondria, heavy and light membranes and cytoplasm.

In some embodiments, sample preparation comprises labeling proteins, polypeptide fragments, or other molecules in a sample with one or more detectable labels. Labeling of a protein or polypeptide fragment may comprise incorporation of a detectable label at the C-terminus, N-terminus, or at one or more interior amino acid residues. Non-limiting examples of labels include biotin, avidin, streptavidin, fluorophors, enzymatic labels, and radiolabels.

Prior to contacting a sample or portion of the sample with a binding reagent as described herein, the sample may be treated with one or more agents capable of denaturing and/or solubilizing proteins, such as detergents (ionic and non-ionic), chaotropes and/or reducing agents. Such agents are known in the art.

Under certain circumstances, it may be desirable to remove or minimize abundant proteins present in a sample, for example, by targeted immunodepletion, or other methods known in the art. Generally, such removal (or reduction) occurs prior to contacting with binding reagent(s) as described herein. For example, in a serum sample, pretreatment may comprise antibodies that bind to albumin, immunoglobulin, and/or other abundant proteins.

In some embodiments, it may be desirable to treat the sample with a polysaccharide cleaving agent, for example, to reduce, minimize, and or eliminate glycosylation of sample protein. Removal of any carbohydrate moieties may be accomplished chemically or enzymatically. Examples of polysaccharide cleaving agents include glycosidases, endoglycosidases, exoglycosylases, and chemicals such as trifluoromethanesulfonic acid. Endoglycosidases such as Endoglycosidase H (New England Biolabs, Beverly, Mass.), and Endo Hf (New England Biolabs) are commercially available. These endoglycosidases cleave the chitobiose core of high mannose and some hybrid oligosaccharides from N-linked glycoproteins. Exoglycosidases are also commercially available from vendors such as New England Biolabs and include, beta-N-Acetylhexosaminidase, alpha-1-2-Fucosidase, alpha-1-3,4 Fucosidase alpha-1-2,3 Mannosidase, alpha-1-6 Mannosidase, Neuraminidase, alpha-2-3 Neuraminidase, beta 1-3 Galactosidase, and alpha-N-Acetyl-galactosaminidase

Biomarkers

Biomarker protein(s) can be characterized using binding reagents as described herein. For example, presence, absence, or amount of a biomarker may be determined by detection of binding to a binding reagent as described herein. A biomarker is a protein or other molecule of interest, for which the detection, monitoring, quantitation, and/or characterization is of interest. In some embodiments, a biomarker is correlated with a specific condition or treatment, such as a disease or condition, treatment with a drug (including efficacy of drug treatment and/or toxicity), treatment with a medical device, and the like. In other embodiments, a biomarker is expressed in a tissue or cell of interest (e.g., a tumor, an organ, etc.). In one embodiment, a biomarker is a protein or protein variant (such as a mutant protein, splice variant, a protein with altered post-translational modification, etc.). In some embodiments, a biomarker is a tissue-specific marker.

A biomarker can be used as a surrogate marker in diagnosis (including staging of disease, in some embodiments), prognosis, evaluation and/or selection of therapies, monitoring of disease progression, monitoring of efficacy of treatment, and/or treatment of disease. In some embodiments, a biomarker is detected and/or quantified by detection of binding to a binding reagent as described herein, whereby expression of the biomarker (presence or absence of biomarker, or differential expression of the biomarker) indicates the presence of a disorder or a condition. In one embodiment, increase in level of a biomarker indicates the presence of a disorder or condition. In another embodiment, decrease in level of a biomarker indicates the presence of a disorder or condition. In some embodiments, biomarker expression is used to evaluate the efficacy of a particular therapeutic treatment regimen in animal studies, in clinical trials, or to monitor the treatment of an individual subject. In some embodiments, the biomarker serves as a proxy for a desired clinical endpoint. In other embodiments, the biomarker is correlated with efficacy of an agent, as when biomarker expression is predictive of efficacy of treatment with an agent (such as a drug). In one embodiment, increase in level of a biomarker indicates efficacy or progress of treatment. In another embodiment, decrease in level of a biomarker indicates efficacy or progress of treatment.

A biomarker may be a marker for toxicity, including, for example, toxicity of an agent such as a pharmaceutical, new drug candidate, cosmetic, or other chemical. In some embodiments, detection and/or quantitation of biomarker expression may be used to monitor for environmental exposure to an agent, such as a toxin or a pathogen. In one embodiment, presence or increase in level of a biomarker indicates toxicity or exposure to an agent. In another embodiment, absence or decrease in level of a biomarker indicates toxicity or exposure to an agent.

In some embodiments, one or more binding reagents as described herein that specifically binds a biomarker can be used for the detection of the biomarker (including in vitro and in vivo detection). In one embodiment, a binding reagent that specifically binds a biomarker can be linked to an in vivo imaging reagent, such as, for example, ³H, ¹¹¹In, ¹²⁵I, (see Esteban et al. (1987) J. Nucl. Med. 28.861-870), and used in an in vivo imaging application.

Compositions and Kits

The invention provides compositions comprising one or more binding reagents as described herein.

In some embodiments, compositions are provided comprising a plurality of binding reagents, wherein each different binding reagent recognizes a different molecule. In some embodiments, compositions are provided comprising a plurality of binding reagents, each of which comprises a unique detectable label.

In one embodiment, the invention provides a composition comprising a protein binding reagent, wherein the protein binding reagent comprises a plurality of linked small epitope protein binding molecules each recognizing a different small epitope consisting of 3 to 5 amino acids. In one embodiment, the small epitope protein binding molecules are small epitope antibodies.

In some embodiments, the invention provides a pharmaceutical composition comprising a binding reagent as described herein and a pharmaceutically acceptable carrier. As used herein, a “pharmaceutically acceptable carrier” refers to a carrier that is biocompatible (i.e., not toxic to the host) and suitable for a particular route of administration for a pharmacologically effective substance. Suitable pharmaceutically acceptable carriers include but are not limited to stabilizing agents, wetting and emulsifying agents, salts for varying osmolarity, encapsulating agents, buffers. Examples of pharmaceutically acceptable carriers are described in Remington's Pharmaceutical Sciences (Alfonso R. Gennaro, ed., 18th edition, 1990). A pharmaceutical composition of the invention may comprise a therapeutically effective amount of a binding reagent for administration to an individual in need of treatment for a condition for which such administration may be therapeutically beneficial. A “therapeutically effective amount” or “therapeutic dose” refers to an amount of a binding reagent sufficient to effect beneficial clinical results, such as for example reduction or alleviation of a symptom of a disease, reduction in tumor growth, etc.

In another aspect, the invention includes compositions comprising intermediates (such as complexes, e.g., protein binding reagent-protein complex) produced by any aspect of the methods of the invention. The invention also provides incubation mixtures comprising samples and binding reagents and/or binding reagent-molecule of interest complexes, as described herein.

The invention also provides kits for use in the instant methods. Kits of the invention may include one or more containers each comprising one or more binding reagents as described herein. A kit may comprise any of about 2, 3, 5, 10, 20, 30, 40, 50, 75, 100, 125, 150, 200, 300, 400, 500, 1000, or more binding reagents. In some embodiments, a kit comprises a plurality of binding reagents containing any of at least about 2, 3, 5, 10, 20, 30, 40, 50, 75, 90, 100, 125, 150, 200, 300, 400 or 500, with an upper limit of any of about 10, 20, 30, 40, 50, 75, 100, 125, 150, 200, 300, 400, 500, or 1000 binding reagents.

In some embodiments, one or more binding reagents in a kit of the invention are detectably labeled. In some embodiments, the kits include a plurality of binding reagents each labeled with a unique detectable label.

In some embodiments, a kit further comprises instructions for use in accordance with any of the methods of the invention described herein, such as detection, quantitation, and/or purification of a molecule of interest, or methods of treatment of a disease or targeting of a therapeutic agent to an in vivo site of action. Instructions may be provided in printed form, on magnetic media, such as a CD or DVD, or in the form of a website address at which the instructions may be obtained.

The kits are in suitable packaging. Suitable packaging includes, but is not limited to, vials, bottles, jars, flexible packaging (e.g., sealed Mylar or plastic bags), and the like. In some embodiments, the kit comprises a container and a label or package insert(s) on or associated with the container. The label or package insert may indicate that the binding reagent(s) is(are) useful for any of the methods described herein. Instructions may be provided for practicing any of the methods described herein.

In one embodiment, the invention provides a kit comprising a library of binding reagents, each of which contains a plurality of small epitope binding molecules which recognizes a different combination of small epitopes.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entireties for all purposes and to the same extent as if each individual publication, patent, or patent application were specifically and individually indicated to be so incorporated by reference.

We claim:

1. A binding reagent comprising a plurality of small epitope binding molecules, wherein said small epitope binding molecules are linked, and wherein each of said small epitope binding molecules recognizes a different small epitope.

2. A binding reagent according to claim 1, wherein said plurality of small epitope binding molecules consists of 2, 3, or 4 small epitope binding molecules.

3. A binding reagent according to claim 1, wherein said small epitope binding molecules are small epitope antibodies or epitope binding fragments thereof.

4. A binding reagent according to claim 3, wherein each small epitope antibody or epitope binding fragment thereof recognizes an epitope consisting of 3, 4, or 5 contiguous amino acids.

5. A binding reagent according to claim 3, wherein said plurality of small epitope binding molecules consists of 2, 3, or 4 small epitope antibodies or epitope binding fragments thereof.

6. A binding reagent according to claim 1, wherein said small epitope binding molecules are selected from the group consisting of aptamers, molecular imprints, lectins, and capture compounds.

7. A binding reagent according to claim 1, wherein the binding reagent binds specifically to a protein.

8. A binding reagent according to claim 1, wherein the small epitope binding molecules are linked covalently.

9. A binding reagent according to claim 8, wherein the covalent linkage comprises a chemical cross-linking reagent.

10. A binding reagent according to claim 1, wherein the small epitope binding molecules are linked by interaction between members of a binding pair.

11. A binding reagent according to claim 3, wherein the reagent is recombinantly expressed as a polypeptide with a peptide linker between the antibodies or epitope binding fragments.

12. A binding reagent according to claim 1, comprising a detectable label.

13. A library of binding reagents, comprising a plurality of binding reagents according to claim 1, wherein each of said binding reagents recognizes a different combination of small epitopes.

14. A method of characterizing a molecule of interest, said method comprising contacting a sample suspected of comprising the molecule of interest with a binding reagent according to claim 1, wherein said binding reagent is capable of specifically binding to the molecule of interest, and detecting binding of the molecule of interest to the binding reagent.

15. A method according to claim 14, wherein characterizing comprises determining the amount of the molecule of interest in the sample.

16. A method according to claim 14, wherein characterizing comprises determining presence or absence of the molecule of interest in the sample.

17. A method according to claim 14, wherein the molecule of interest is a biomarker.

18. An array comprising a plurality of binding reagents according to claim 1 immobilized on a support, wherein each of said binding reagents recognizes a different combination of small epitopes.

19. A method of making a binding reagent according to claim 1, comprising linking said small epitope binding molecules with a chemical cross-linking reagent.

20. A method of making a binding reagent according to claim 1, comprising linking said small epitope binding molecules via interaction between members of a binding pair.

21. A method of making a binding reagent according to claim 3, comprising recombinant expression of a polypeptide comprising said small epitope antibodies or epitope binding fragments thereof.

22. A kit comprising a binding reagent according to claim 1.

23. A composition comprising a binding reagent according to claim 1.

24. A pharmaceutical composition comprising a binding reagent according to claim 1 and a pharmaceutically acceptable carrier.

25. An affinity matrix comprising a binding reagent according to claim 1 attached to a substrate.

26. A method for purifying a molecule of interest, comprising contacting an affinity matrix according to claim 25 with a sample comprising the molecule of interest, wherein said binding reagent binds to said molecule of interest.

27. A method according to claim 26, wherein said molecule of interest is selected from the group consisting of a polypeptide, a polynucleotide, a polysaccharide, and a glycoprotein.
